(19) United States
(12) Patent Application Publication
(10) Pub. No.: US 2003/0050644 A1
(43) Pub. Date: Mar. 13, 2003

(54) Systems and methods for accessing and treating diseased or fractured bone employing a guide wire

(76) Inventors: Ryan P. Boucher, San Francisco, CA (US); Richard W. Layne, Sunnyvale, CA (US); Nenita Salom, Tracy, CA (US); Lulu Follmer, Santa Clara, CA (US); Alberto Ruiz Cantu, Fremont, CA (US); Karen D. Talmadge, Palo Alto, CA (US); Robert M. Scribner, Niwot, CO (US)

Correspondence Address: RYAN KROMHOLZ & MANION, S.C., Post Office 26618, Milwaukee, WI, 53226-0618, US

(21) Appl. No.: 09/952,014

(22) Filed: Sep. 11, 2001

(51) Int. Cl. A61B 17/58

(52) U.S. Cl. 606/90

(57) ABSTRACT

Systems and methods for treating bone employ an expandable body sized to be inserted into bone over a guide wire, without need of an access cannula, and undergo expansion in cancellous bone to compact cancellous bone. The systems and methods further include one or more other instruments sized to pass over the guide wire. The other instruments can comprise, e.g., a cannula, or a device for injecting material into bone. The systems and methods can make use of a special guide wire assembly that includes a guide wire having an enlarged component or tip element on its distal end that can be used to engage the distal end of a bone treatment tool in response to a pulling motion on the guide wire. The pulling motion on the guide wire serves to withdraw the bone treatment tool. The enlarged component or tip element on the distal end of the guide wire can enable the withdrawal of an inner centering body from an outer cannula body after deployment. After withdrawal of the inner body, the outer body can be used, e.g., to guide a bone treatment tool into a bone, or to convey material into bone

### FIELD OF THE INVENTION

This invention relates to the treatment of bone conditions of the human and other animal body systems and, more particularly, to systems and methods for correcting such conditions.

### BACKGROUND OF THE INVENTION

Vertebroplasty and Kyphoplasty are two minimally-invasive procedures that have been developed to access and treat diseased or fractured bone, such as collapsed or fractured vertebral bodies in individuals suffering from osteoporosis. In a vertebroplasty procedure, poly(methylmethacrylate) (PMMA) or bone cement (such as Simplex-P® commercially available from Howmedica) is injected directly into the interior of a weakened and/or fractured bone in an attempt to reinforce the bone and prevent further fracture. In a Kyphoplasty procedure, a surgeon manipulates the cancellous and/or cortical bone of the weakened and/or fractured vertebral body with surgical tools, and then introduces a void filler such as bone cement into the bone, desirably into a cavity formed within the vertebral body, in an attempt to repair, reinforce and/or prevent further fracture or subsidence of the bone.

Both of these procedures seek to reduce the pain and discomfort experienced by patients suffering from vertebral compression fractures, and both procedures seek to reinforce a fractured and/or weakened vertebral body against further fracture. The Kyphoplasty procedure additionally permits a practitioner to reduce or repair the fractured bone prior to fixation—in a manner similar to setting a broken arm or leg bone to a more normal anatomical position before applying a cast to the damaged extremity—and allows creation of a cavity within the vertebral body to contain the filler material. Desirably, the filler material will form an “internal cast” to support the vertebral body against further loading. Desirably, the Kyphoplasty technique permits a practitioner to restore the anatomy and loading of the spine to a pre-fractured condition, and also minimizes the opportunities for extravazation or leakage of the filler material outside of the targeted bone.

Both of these techniques are desirably minimally-invasive, and both generally employ a substantially rigid hollow access tool or cannula having an interior lumen through which the interior of the bone is accessed. These access tools, which are typically designed to penetrate rigid tissue such as cortical bone, generally require significant column strength to penetrate and transit the rigid tissue and are thus essentially non-expandable. Consequently, the size of the inner lumen of such access tools basically defines the maximum dimensions of any therapeutic substance and/or surgical tools that can pass through the access tool into the vertebral body.

Because a vertebroplasty procedure entails only the injection of a flowable material, such as bone cement, into the fractured vertebral body, the lumen of the access tool necessary for introduction of such substances can be rather small. A common access tool used in a vertebroplasty procedure is an 11-gage spinal needle having an outer diameter of 0.120 inches and an interior lumen approximately 0.095″ in diameter. Because this tool is of such small diameter, it desirably causes very little soft tissue and/or bone trauma and can be inserted through smaller access paths, such as through the pedicles in the vertebral bodies of the cervical and upper lumbar regions of the human spine.

In contrast, a Kyphoplasty procedure employs tools, such as inflatable bone tamps, to manipulate the cancellous bone and/or move the cortical bone. These tools generally require a somewhat larger access path than that required for a typical vertebroplasty procedure. An access tool suitable for use in a Kyphoplasty procedure might be approximately the size of an 8-gage or larger needle assembly. Such larger tools, however, can potentially cause additional soft tissue and/or bone damage and might be unsuitable for insertion through smaller access paths, such as through the pedicles in the vertebral bodies of the cervical and upper lumbar regions of the human spine.

Because a Kyphoplasty procedure permits a surgeon to reduce the fractured vertebral body and/or compress weakened cancellous bone prior to fixation, and permits creation of a cavity within the vertebral body for the filler material, a Kyphoplasty procedure has numerous clear advantages over a vertebroplasty procedure. There is a need, therefore, for a procedure which permits manipulation of the cancellous/cortical bone and/or creating of a cavity within the cancellous bone, but which provides for a smaller, less invasive access path through the soft tissue and/or into the vertebral body.

### SUMMARY OF THE INVENTION

One aspect of the invention provides systems and methods that make use of a special guide wire assembly. The guide wire assembly includes a guide wire which incorporates a distal end having an outside diameter. The guide wire desirably includes a component or tip element that extends beyond the distal end. The guide wire component or tip element has an enlarged outside diameter that is greater than the outside diameter of the distal end of the guide wire itself. The guide wire assembly can be used, e.g., to guide deployment of a bone treatment tool through soft tissue and inside bone, without need of an access cannula. The bone treatment tool can, for example, carry an expandable structure that, when deployed inside bone, compacts cancellous bone, e.g., to create a cavity or to move cortical bone. According to another aspect of the invention, the enlarged component or tip element on the distal end of the guide wire can be used to engage the distal end of the bone treatment tool in response to a pulling motion on the guide wire. The pulling motion on the guide wire serves to withdraw the bone treatment tool. This aspect of the invention allows a bone treatment tool with a damaged or parted distal end portion to be retrieved.

According to another aspect of the invention, systems and methods described herein provide a bone access assembly usable in association with the guide wire assembly, as just described. The bone access assembly includes an outer body and an inner body. The inner body is sized to occupy an interior lumen of the outer body. The inner body has an interior passage sized to pass over the guide wire. In use, the enlarged component or tip element on the distal end of the guide wire engages the distal end of the inner body in response to a pulling motion on the guide wire. The pulling motion on the guide wire serves to move the inner body proximally through the outer body for removal from the interior lumen. In this arrangement, the inner body serves to center the outer body over the guide wire, and, further, the guide wire serves to withdraw the inner body after desired deployment of the outer body into the bone has been accomplished. The outer body can be used, e.g., as a cannula to guide a bone treatment tool into a bone, or to directly convey material into the bone.

According to another aspect of the invention, systems and methods described herein provide a cannula and/or bone filler assembly usable in association with the guide wire assembly, the cannula and/or bone filler assembly adapted to minimize trauma to the bone tissue.

The present methods and devices also desirably permit the practitioner to reduce the complexity of a Kyphoplasty procedure. When using an access tool or cannula as the primary access path during the entire Kyphoplasty procedure, a number of “tool exchanges” is typically required, each exchange usually adding to the total time required to complete the surgical procedure. For instance, when utilizing an expandable structure to treat a collapsed and/or fractured vertebral body through an access tool or cannula, a physician will typically (1) insert a spinal needle assembly into the vertebral body; (2) withdraw the needle stylet; (3) insert a tracking stylet or “K-Wire” and withdraw the spinal needle; (4) insert a blunt obturator and withdraw the tracking stylet; (5) insert a cannula and withdraw the blunt obturator; (6) insert a drill, drill a channel, and withdraw the drill; (7) insert an expandable structure, expand and contract the structure and then remove the structure; and (8) fill the cavity. In contrast, the teachings of the present invention allow a surgeon to accomplish an equivalent procedure with fewer steps, for example: (1) inserting a spinal needle and guide wire assembly into the vertebral body; (2) removing the spinal needle; (3) inserting the expandable tool along the guide wire, expanding and contracting the structure and then remove the structure; and (4) filling the cavity.

Accordingly, another aspect of the invention provides systems and methods for treating bone. The systems and methods employ an expandable body sized to be inserted into bone over a guide wire, without need of an access cannula, and undergo expansion in cancellous bone to compact cancellous bone.

In one embodiment, the systems and methods further include another instrument sized to pass over the guide wire. The other instrument can comprise, e.g., a cannula, or a device for injecting material into bone.

In one embodiment, the systems and methods form a cavity in cancellous bone by expansion of the expandable body.

Other features and advantages of the inventions are set forth in the following Description and Drawings, as well as in the appended Claims.

### BRIEF DESCRIPTION OF THE DRAWINGS

The drawings are not intended to be true anatomic views, but serve to illustrate the various aspects of the invention.

FIG. 1 is a top view of a human vertebral body;

FIG. 2 is a side view of a human vertebra;

FIG. 3 is a plane view showing a kit containing a system of instruments used to treat bones and that embodies features of the invention;

FIG. 4 is a perspective view of a spinal needle assembly that is contained in the kit shown in FIG. 3, including a spinal needle, a guide wire, and a guide wire component;

FIG. 4a is a perspective view of one embodiment of a guide wire component extending beyond a distal end of a guide wire, showing the guide wire component constructed in accordance with the teachings of the present invention;

FIG. 4b is a perspective view of another alternate embodiment of a guide wire component extending beyond a distal end of a guide wire, showing the guide wire component constructed in accordance with the teachings of the present invention;

FIG. 5 is a side sectional view of a bone compaction device that is contained in the kit shown in FIG. 3, including a catheter tube assembly, a y-shaped adaptor handle, and an expandable structure;

FIG. 6a is a side sectional view of a bone filling device that is contained in the kit shown in FIG. 3, showing an outer body and an inner body, and further showing a guide wire assembly in phantom;

FIG. 6b is a side sectional view of the bone filling device of FIG. 6a, showing the outer body and the inner body, and further showing the guide wire assembly within the inner body;

FIG. 6c is a side sectional view of the bone filling device that is contained in the kit shown in FIG. 3, showing an outer body and further showing a syringe that is contained in the kit shown in FIG. 3 attached in phantom;

FIG. 6d is a side sectional view of the outer body of the bone filling device, showing a tamp that is contained in the kit shown in FIG. 3 contained partially within the outer body;

FIG. 7 is a top view showing the spinal needle assembly being inserted into a vertebral body

FIG. 8 is a top view showing the spinal needle being withdrawn from the guide wire assembly and from the vertebral body;

FIG. 9 is a top view showing the bone compaction device inserted along the guide wire into the vertebral body;

FIG. 10 is a top view showing the bone compaction device of FIG. 9 with a syringe attached thereto and expansion of the expandable structure compressing cancellous bone and/or moving cortical bone;

FIG. 11 is a top view showing the guide wire assembly and an interior cavity created by expanding the expandable structure;

FIG. 12 is a top view showing the bone filling device including the outer body and the inner body inserted over the guide wire and into the cavity in the vertebral body;

FIG. 13 is a top view showing removal of the guide wire assembly and the inner body from the outer body of the bone filling device by pulling on the guide wire;

FIG. 14a is a top view showing the syringe attached to the outer body of the bone filling device and partial delivery of a bone filling material into the cavity;

FIG. 14b is a top view showing gradual withdrawal of the outer body of FIG. 14a as the bone filling material fills the cavity;

FIG. 14c is a top view showing the outer body of the bone filling device nearly withdrawn from the interior cavity and the bone filling material fully occupying the cavity;

FIG. 15 is a top view showing an outer body of a bone filling device within an optional cannula, the cannula secured to an outer cortical wall of the vertebral body and the distal end of the guide wire located at the far side of the vertebral body;

FIG. 16 is a top view showing the optional cannula of FIG. 15 with a physician pulling on the guide wire to remove the guide wire assembly from the optional cannula;

FIG. 17 is a top view showing the bone compaction device over the guide wire and the expandable structure within the interior cavity, the proximal end of the expandable structure having torn away from a catheter tube assembly; and

FIG. 18 is a top view showing the torn expandable structure and bone compaction device being removed by pulling on the guide wire;

FIG. 19 is a top view of one embodiment of a low profile cannula constructed in accordance with the teachings of the present invention;

FIG. 20 is a top view of one embodiment of a low profile bone filling device constructed in accordance with the teachings of the present invention.

The invention may be embodied in several forms without departing from its spirit or essential characteristics. The scope of the invention is defined in the appended claims, rather than in the specific description preceding them. All embodiments that fall within the meaning and range of equivalency of the claims are therefore intended to be embraced by the claims.

### DETAILED DESCRIPTION OF THE PREFERRED EMBODIMENTS

The preferred embodiment describes improved systems and methods that embody features of the invention in the context of treating bones. This is because the new systems and methods are advantageous when used for this purpose. However, aspects of the invention can be advantageously applied for diagnostic or therapeutic purposes in other areas of the body.

The new systems and methods will be more specifically described in the context of the treatment of human vertebrae. Of course, other human or animal bone types can be treated in the same or equivalent fashion. By way of example, and not by limitation, the present systems and methods could be used in any bone having bone marrow therein, including the radius, the humerus, the vertebrae, the femur, the tibia or the calcaneus.

I. Anatomy of a Vertebral Body

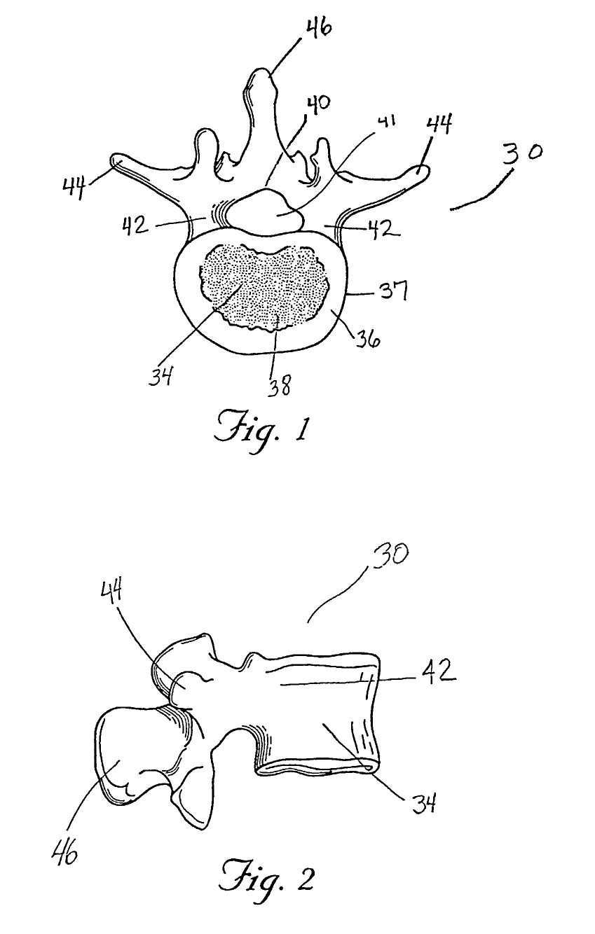

FIG. 1 shows a coronal (top) view of a human lumbar vertebra 30. FIG. 2 shows a lateral (side) view of the vertebra 30. The vertebra 30 includes a vertebral body 34, which extends on the anterior (i.e., front or chest) side of the vertebra 30. The vertebral body 34 gives strength to the spine and supports body weight. The vertebral body 34 is shaped generally like a hockey puck.

The vertebral body 34 includes an exterior formed from compact cortical bone 36. The cortical bone 36 encloses an interior volume of reticulated cancellous 38, or spongy, bone (also called medullary bone or trabecular bone).

The spinal canal 41 is located on the posterior (i.e., the back) side of each vertebra 30. The spinal cord (not shown) passes through the spinal canal 41. The vertebral arch 40 surrounds the spinal canal 41. Left and right pedicles 42 of the vertebral arch 40 adjoin the vertebral body 34. The spinous process 46 extends from the posterior of the vertebral arch 40, with the left and right transverse processes 44 extending from the sides of the vertebral arch 40.

It may be indicated, due to disease or trauma, to compress cancellous bone 38 within the vertebral body 34. The compression, for example, can be used to form an interior cavity which receives a filling material, e.g., allograft tissue, autograft tissue, hydroxyapatite, synthetic bone substitute and/or a flowable material that sets to a hardened condition such as polymeric cements and/or mineral cements, as well as a medication, or combinations thereof, to provide improved interior support for cortical bone or other therapeutic functions, or both. It may also be desired to exert force upon the interior of the cortical bone 36, either directly or in combination with compression of the cancellous bone 38, making it possible to elevate or push broken and compressed bone back to or near its original pre-fracture, or other desired, condition.

Alternatively, it may be indicated to move cortical bone 36 without concurrent compaction of cancellous bone 38. The present system and methods can be utilized to directly and/or indirectly displace cortical bone 36 in one or more desired directions.

II. Vertebral Body Collapse and Compression Fractures

The systems and methods of the present invention are especially suited for treating the collapse and/or compression fractures of vertebral bodies 34. Vertebral body collapse and compression fractures are often noted in individuals with osteoporosis as well as other diseases such as osteopenia or myeloma (bone cancer).

Osteoporosis is a disease of the bone that is most commonly found in the middle-aged and elderly, particularly women. It is characterized by a gradual loss or demineralization of spongy cancellous bone 38, causing the remaining bone to become brittle and lose elasticity, thus rendering the bone weaker and more prone to fracture.

In contrast to cancellous bone 38, cortical bone 36 tissue is much harder and denser. Cortical bone 36 provides a protective layer and support for bones such as vertebral bodies 34. However, where osteoporosis has significantly weakened the cancellous bone 38, the cortical bone may be similarly affected and/or is unable to solely support the loads placed on the spine, and thus the vertebral bodies 34 become especially prone to collapse and/or fracture.

III. Instruments

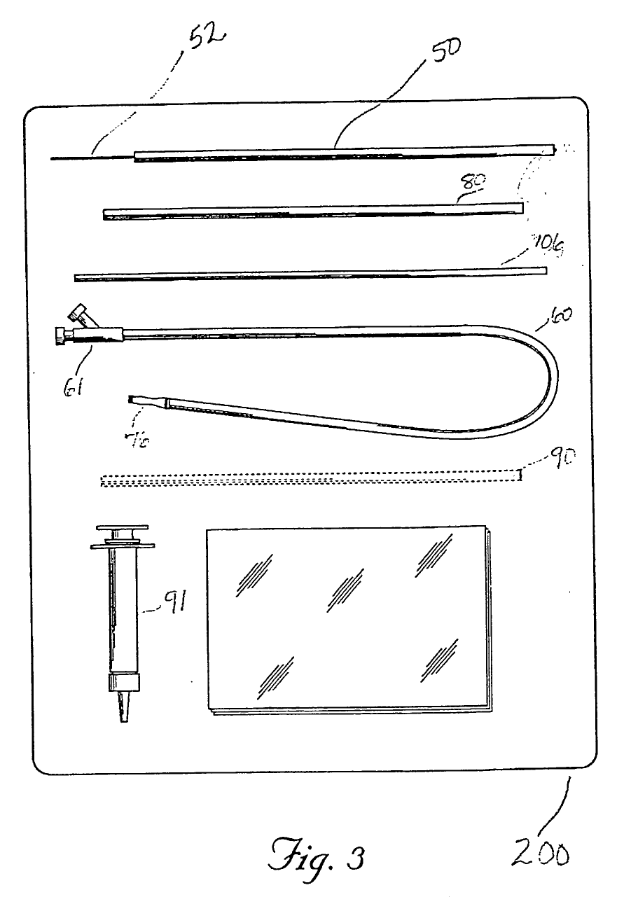

FIG. 3 shows instruments, arranged as a kit 200, which are usable in association with each other to treat diseased bone and to reduce fractured bone. The number and type of instruments can vary. FIG. 3 shows six representative instruments, each having a different size and function.

In FIG. 3, the kit 200 includes a spinal needle assembly 50 that can be used for initially accessing bone; a bone compaction device 60 that can function to create an interior cavity in bone and that carries an expandable structure that may be expanded within the bone; a bone filling device 80 that can function to deliver a bone filling material into an interior cavity in bone; a syringe 91 that can be used for delivering the bone filling material into the bone filling device and/or to expand the expandable structure; a tamp 106 that can function to urge residual bone filling material into bone; and an optional cannula 90 that may be used in combination with a smaller bone filling device to deliver a bone filling material into bone. Instructions for using the kit 200 can also be provided.

A. The Spinal Needle Assembly

The first instrument is a spinal needle assembly, which can be used to initially establish an access path through soft tissue and into bone such as a vertebral body 34.

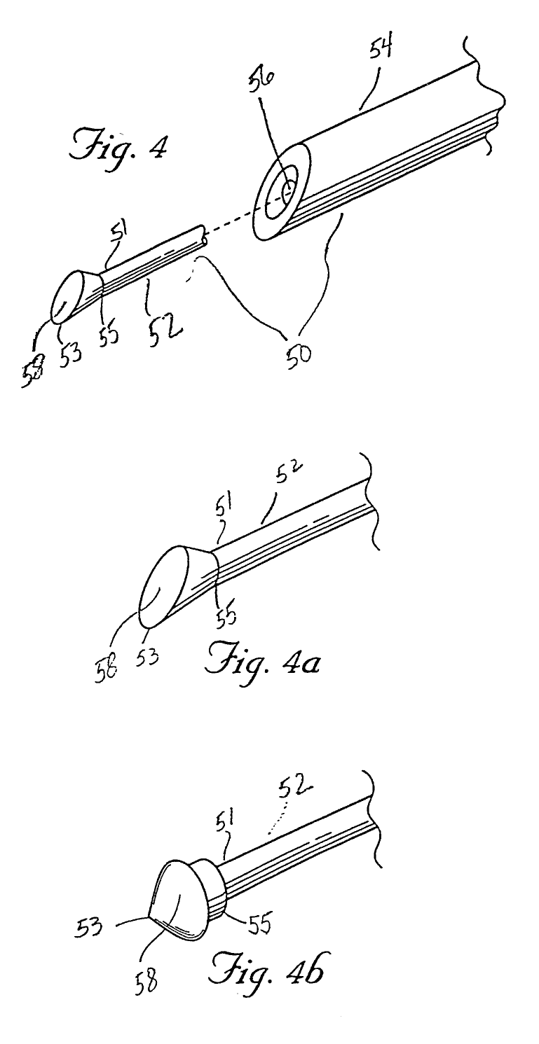

As shown in FIG. 4, the spinal needle assembly 50 desirably includes a guide wire 52 and a spinal needle 54 having a lumen 56 through which the guide wire 52 may pass. In one embodiment, the spinal needle 54 is an 11 gauge spinal needle and the guide wire 52 is a stainless steel wire having a diameter of approximately 0.015 inches. Of course, it should be understood that other sizes and lengths of needles and guide wires, including, but not limited to, 6, 8, 10 or 14 gage spinal needles and guide wires, including those having 0.041 or 0.062 inch diameters, could be used depending upon the location and/or type of bone to be treated. Similarly, the needles and guidewires could be comprised of various surgical materials well known in the art, such as plastic, metal or ceramics.

As will be described later, the guide wire 52 may serve multiple functions. First, the guide wire 52 may be used to guide other instruments to the treatment site. Second, the guide wire 52 may act as a centering device to center other instruments, thereby facilitating their insertion through the access path previously created in the targeted bone. Third, the guide wire 52 may be used to withdraw instruments from the treatment site.

The guide wire 52 has a proximal end 57 and a distal end 51. The distal end 51 has an outside diameter. Extending beyond the distal end 51 is a structure 58 or tip component. If desired, the guide wire can be rigid or flexible, and can incorporate a flexible and/or steerable tip.

Two different representative types of structures 58 are shown in FIGS. 4a and 4b for the purpose of illustration. The structure 58 may be an integral part of the guide wire 52, or it may be attached to the guide wire 52 by welding, gluing or the like. The structure 58 desirably has an enlarged outside diameter that is greater than the outside diameter of the distal end 51 of the guide wire. The structure 58 has a distal surface 53 as well as a proximal surface 55, and these surfaces 53 and 54 are each desirably contoured.

In the embodiment shown in FIG. 4a, the distal surface 53 of the structure 58 desirably forms a portion of the distal tip 58 of the spinal needle assembly 50 which advances through soft tissue (not shown) and into the targeted bone (not shown). Upon withdrawal of the spinal needle 54, however, the distal surface 53 desirably presents a relatively blunt surface to the cancellous bone within the bone, thereby resisting further movement within the vertebral body (not shown). In this embodiment, the distal surface 53 has a relatively blunt shape that is contoured and non-traumatic, and the proximal surface 55 is conical. Desirably, if a longitudinal force acts on the guide wire, such as when a tool is being advanced along the guide wire, the distal surface 53 will tend to contact cancellous or cortical bone (not shown) and will resist further anterior movement within the vertebral body. If desired, the contour of the distal surface 53 can be similar to or different from the contour of the proximal surface 55.

Preferably, the proximal surface 55 of the structure 58 will engage other system components to impart movement to the components in response to the application of pulling forces, as will be described in greater detail later.

B. The Bone Compaction Device

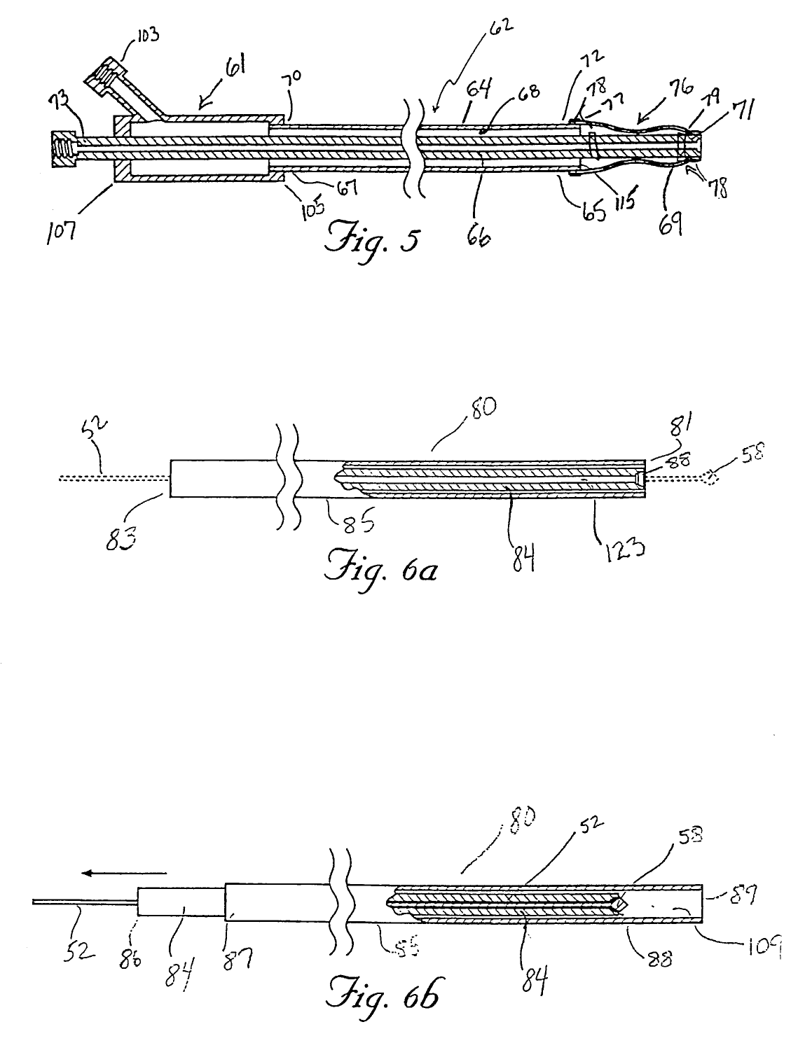

The next instrument is a bone compaction device which functions to compress cancellous bone 38, to elevate cortical bone 36 to an anatomic position, and/or to create a cavity within the targeted bone. If desired, the bone compaction device 60, best shown in FIGS. 3, 5, 9, and 10, can be introduced over the guide wire 52 without use of a cannula or other form of a percutaneous access sheath. In one embodiment, the length of the guide wire is greater than the length of the bone compaction device, which permits the practitioner to manipulate and/or secure the guide wire while the bone compaction device is deployed and/or removed from the targeted bone.

The bone compaction device 60 can be constructed in various ways. In the illustrated embodiment, the bone compaction device 60 comprises a catheter tube assembly 62, a y-shaped adapter 61, and an expandable structure 76.

In the illustrated construction, the catheter tube assembly 62 includes an outer catheter tube 64 and an inner catheter tube 66 that extends through the outer catheter tube 64. The catheter tube assembly 62 desirably defines a flow passage 68 between the outer catheter tube 64 and the inner catheter tube 66.

The catheter tube assembly 62 has a proximal end 70 and a distal end 72. The proximal end 70 of the catheter tube assembly 62 is coupled to the distal end 105 of the y-shaped adapter 61, which thereby serves as a handle and inflation port for the device 60. The distal end 72 of the catheter tube assembly 62 is coupled to the expandable structure 76.

The y-shaped adapter 61 has an interior passage through which fluid, such as an expansion fluid for the expandable structure 76, can pass. The adapter 61 has a port 103 through which an expansion fluid (such as Conray® solution commercially available from Mallinkrodt, Inc.) may be introduced. A syringe 91 or other device may be coupled to the port 103 to deliver the expansion fluid to the expandable structure 76. The expansion fluid can pass from the port 103 through the flow passage 68 and into an expandable structure 76. The expandable structure 76 receives the expansion fluid and inflates or expands as the expansion fluid fills the expandable structure 76. In so doing, the expandable structure 76 may compress cancellous bone 38, compact or lift cortical bone 36, and/or create a cavity within bone.

In one embodiment, the inner catheter tube 66 is made of TEXIN® polyurethane extruded over a stainless steel hypodermic tube 65. In this embodiment, the outer diameter of the inner catheter tube 66 is approximately 0.032 inch, and the stainless steel hypodermic tube 65 of has an outer diameter of approximately 0.025 inch and an inner diameter of approximately 0.020 inch. Desirably, the inner catheter tube 66 carries one or more iridium or platinum radio-opaque marker bands 78, which function to locate the expandable structure 76 using radiologic or other monitoring. The distal tip of the inner catheter tube 66 is desirably open, which permits the inner catheter tube 66 to pass over the guide wire 52. Of course, if desired the inner catheter could comprise a flexible plastic material, thereby increasing the flexibility of the inner catheter.

In one embodiment, the outer catheter tube 64 has an outer diameter of about 0.082 inch and an inner diameter of about 0.042 inch, with a length of approximately 235 mm. The expandable structure 76 has an outer diameter (not expanded) of about 0.065 inch and an inner diameter (not expanded) of about 0.046 inch, with a length of about 15 to 20 mm.

The outer catheter tube 64, inner catheter tube 66, and the expandable structure 76 can be formed generally from the same types of materials, such as, e.g., medical grade metals, plastics, and/or ceramics, including (but not limited to) stainless steel, titanium, polyethylene, polyurethane, latex, rubber, nylon, or Mylar.

Desirably, the inner catheter tube 66 and outer catheter tube 64 have sufficient column strength to permit advancement of the structure 76 along the guide wire, through soft tissues and into the targeted bone. In addition, the inner catheter tube 66 desirably has sufficient tensional strength, and the outer catheter tube 64 has sufficient column strength, to minimize growth of the expandable structure 76 along its longitudinal axis during expansion. For example, if the inner catheter tube 66 has insufficient tensional strength and/or excessive elasticity, it may stretch during expansion of the expandable structure 76, allowing the proximal and distal ends 77 and 79 of the expandable structure 76 to move apart from each other and lengthen the expandable structure 76. Similarly, if the outer catheter tube 64 has insufficient column strength and/or excessive elasticity, it may collapse or deform as the expandable structure 76 expands, resulting in the proximal and distal ends 77 and 79 moving apart from each other and lengthening the expandable structure 76.

Additional details of the device 60 in general and the expandable structure 76 in particular can be found in co-pending U.S. patent application Ser. No. 09/088,459, filed Jun. 1, 1998 and entitled “Expandable Preformed Structures for Deployment in Interior Body Regions”, and co-pending U.S. patent application Ser. No. 09/595,963, filed Jun. 19, 2000 and entitled “Expandable Preformed Structures for Deployment in Interior Body Regions, the disclosures of which are both incorporated herein by reference.

C. The Bone Filling Device

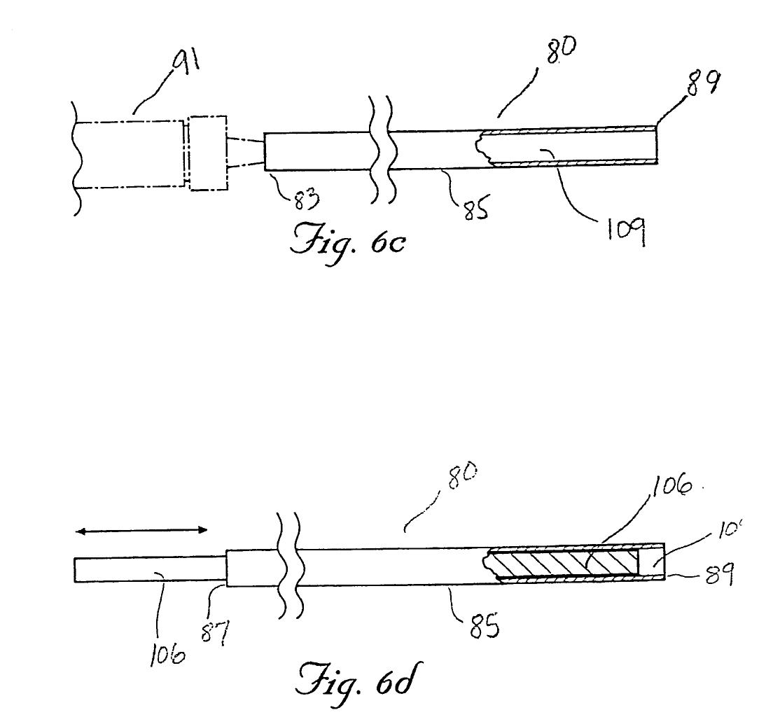

The bone filling device 80 (see FIGS. 6a to 6d) can function to deliver a bone filling material 102 to the bone, either directly to the bone (as in a vertebroplasty-like procedure) or into a cavity previously created within the bone. The bone filling device 80 can be introduced over the guide wire 52 with or without use of a cannula.

In one embodiment, the bone filling device 80 includes an outer body 85 and a inner body 84. The outer body 85 has an interior lumen 109. The outer body 85 has a proximal end 87 and a distal end 89. The proximal end 87 of the outer body 85 desirably includes a fitting 83 that is adapted to couple with a injection device comprising a source of filling material 102 to convey filling material 102 into and/or through the interior lumen 109.

The inner body 84 is desirably sized to occupy the interior lumen 109 (see FIGS. 6a and 6b). The inner body 84 has an interior passage 123 that is sized to pass over the guide wire 52. The inner body 84 has a proximal end 86 and a distal end 88. The inner body 84 desirably functions to center the outer body 85 over the guide wire 52 during deployment of the bone filling device 80 into bone.

The distal end 88 is desirably adapted to engage or mate with the proximal surface of the distal structure 58 of the guide wire 52. The distal end 88 of the inner body 84 may be conical or graduated, or can be any other shape desirably sized and adapted to accommodate a portion or all of the proximal contoured surface of the distal structure 58 of the guide wire 52. Alternatively, the proximal surface of the distal structure 58 may simply abut against the distal end 88. Desirably, the distal end 58 of the guide wire 52 is larger than the interior passage 123, but smaller than the interior lumen 109, such that the guide wire 52 may be used to slidably move or “pull” the inner body 84 proximally through the outer body 85 for removal from the interior lumen 109 (as FIG. 6b shows). Once the guide wire 52 and the inner body 84 are engaged, pulling on the guide wire 52 can remove the inner body 84 from the outer body 85, opening the interior of the outer body 85 to accommodate passage of a filling material 102 (see FIG. 6c), as will be described in greater detail later.

D. The Tamp

As shown in FIG. 6c, the tamp 106 functions to urge residual bone filling material from the outer body 85.

E. The Cannula

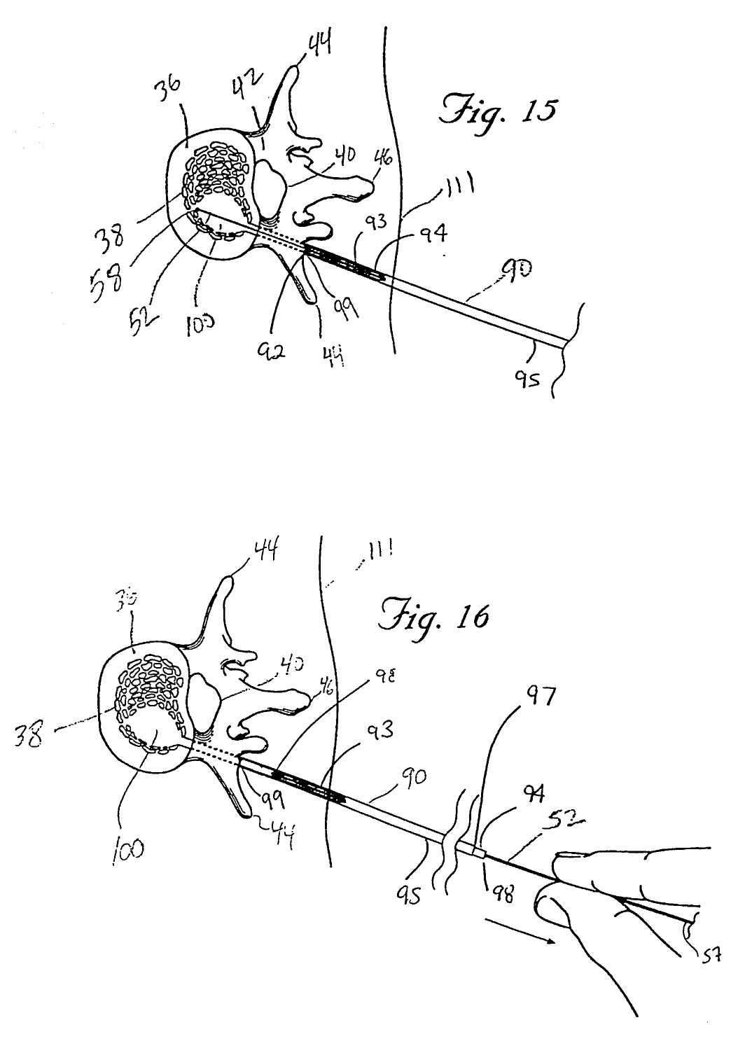

To avoid leakage of the bone filler material out of the bone filler device and into surrounding soft tissues, a cannula 90 (see FIGS. 15 and 16) may be introduced over the guide wire 52 to provide an access path into bone for the bone filling device 80. In the disclosed embodiment, the cannula 90 includes an outer body 95 and an inner body 94. The outer body 95 of the cannula 90 has a proximal end 97 and a distal end 99. The inner body 94 includes an interior lumen 93 and has a proximal end 96 and a distal end 98. The interior passage 93 of the inner body 94 desirably functions to center the outer body 95 over the guide wire 52.

The distal end 98 is desirably adapted to engage or mate with the proximal surface 55 of the guide wire 52. The distal end 98 of the inner body 94 is desirably sized to accommodate a portion or all of the proximal surface 55 of the distal structure 58 of the guide wire 52. As previously noted, the guide wire 52 desirably can be used to slidably enable removal of the inner body 94 from the treatment site.

Once the guide wire 52 and the inner body 94 are engaged, pulling on the proximal end 57 of the guide wire 52 removes the inner body 94 and the guide wire 52 from the outer body 95 (see FIG. 16), opening the interior lumen 93 to accommodate passage 85 of a bone filling device 80. If desired, the cannula 90 may be inserted or imbedded into the cortical bone, or can incorporate teeth 92 or other securing devices which allow the cannula 90 to be secured to the outer surface of cortical bone 36 rather than inserting the cannula 90 into the bone. Alternatively, the cannula 90 may incorporate a sealing mechanism such as an inflatable bladder or o-ring (not shown) near the distal tip which can engage the targeted bone and/or secure the cannula to the targeted bone. Subsequently, a bone filling device may be introduced through the cannula into the bone to convey a bone filler material into the bone, or filler material may be introduced directly through the cannula. If desired, a plurality of bone filling devices may be used to introduce bone filling material into the targeted bone.

In another alternate embodiment, the bone filling device may include a plurality of lumens (not shown), with the guide wire passing through one of the lumens while bone filling material is contained in one or more other lumens. In such an embodiment, the bone filling device may track along the guide wire to the targeted bone treatment site, and then bone filler can be introduced through the other lumen(s).

If desired, the distal tip of the bone filling device may incorporate a seal or frangible tip (not shown) which desirably prevents leakage of the bone filler material during introduction and/or removal of the device from the targeted bone, but which permits introduction of bone filler material to the bone treatment site.

F. Low Profile Tools

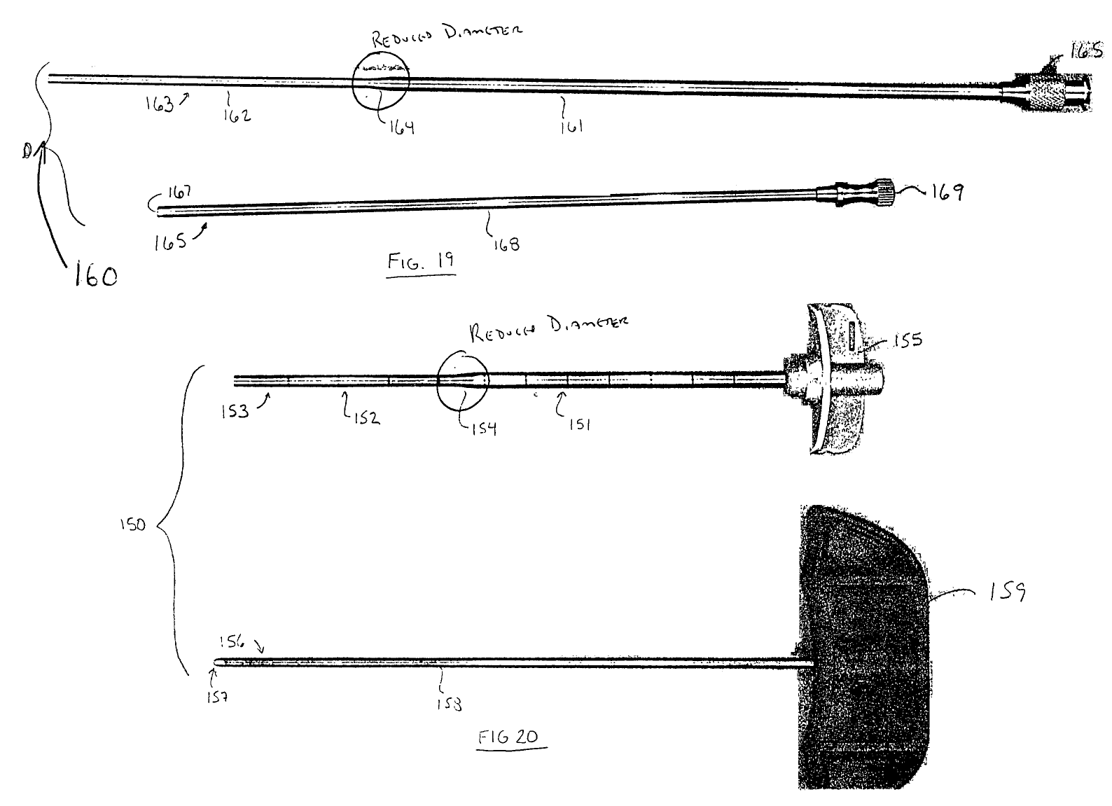

FIGS. 19 and 20 depict an alternate embodiment of an introducer tool 150 and associated bone filler device 160, constructed in accordance with the teachings of the present invention. Both of these embodiments incorporate a distal portion having a reduced profile.

The introducer tool 150 comprises a cannula 153 and a stylet 156. The cannula 153 includes a large diameter portion 151, a small diameter portion 152, a transition portion 154 and a first handle 155. The stylet 155 includes a pointed distal tip 157, a shaft section 158 and a second handle 159. The shaft section 158 is desirably sized to fit within a lumen (not shown) extending through the cannula 153, and may be of a constant or varying size.

The bone filler device comprises a nozzle 163 and a tamp 165. The nozzle 163 includes a large diameter portion 161, a small diameter portion 162, a transition portion 164 and a first handle 165. The tamp 165 includes a blunt distal tip 167, a shaft section 168 and a second handle 169. The shaft section 168 is desirably sized to fit within the lumen (not shown) extending through the nozzle 163, and may be of a constant or varying size. In the disclosed embodiment, the shaft section 168 is sized to fit within a lumen extending through the large diameter portion 161.

For example, one embodiment of a low profile introducer tool could comprise a cannula having a 3.5″ long large diameter portion of approximately 0.180″ outer diameter (OD) by 0.158″ inner diameter (ID), a 2″ long small diameter portion of approximately 0.134″ OD by 0.114″ ID, and a 3/16″ long transition section, with a corresponding 6.25″ long stylet having an outer diameter of approximately 0.107″. A low profile bone filling device suitable for use with such an introducer tool could comprise a nozzle having a 6″ long large diameter portion of approximately 0.148″ OD by 0.126″ ID, a 3″ long small diameter portion of approximately 0.109″ OD by 0.091″ ID, and a 3/16″ long transition section, with a corresponding 6″ long tamp having an outer diameter of approximately 0.111″. Desirably, this tamp would displace approximately 1.2 cc of filler material upon full insertion into the nozzle.

As another example, another embodiment of a low profile introducer tool could comprise a cannula having a 3.5″ long large diameter portion of approximately 0.203″ OD by 0.181″ ID, a 2″ long small diameter portion of approximately 0.134″ OD by 0.114″ ID, and a 3/16″ long transition section, with a corresponding 6.25″ long stylet having an outer diameter of approximately 0.107″. A low profile bone filling device suitable for use with such an introducer tool could comprise a nozzle having a 6″ long large diameter portion of approximately 0.175″ OD by 0.158″ ID, a 3″ long small diameter portion of approximately 0.109″ OD by 0.091″ ID, and a 3/16 long transition section, with a corresponding 6″ long tamp having an outer diameter of approximately 0.151″. Desirably, this tamp would displace approximately 2 cc of filler material upon insertion into the nozzle. Of course, if desired alternate embodiments of the large diameter portion 161 or “reservoir” of the bone filler device could be sized to accommodate various amounts of filler material, such as 0.5 cc, 0.75 cc, 1 cc, 1.5 cc, 3 cc, 4 cc or 5 cc.

Desirably, the reduced distal tip diameter of the cannula 153 and/or bone filling device 160 will allow the tip of these tools to be inserted into the targeted bone, with a corresponding reduction in the size of the access path created in the bone. The smaller diameter section of the tool will pass through the cortical wall into the bone, while the larger diameter section can abut against the outside of the bone (sealing the opening, if desired), and will desirably stretch, but not tear, softer tissues. For example, where the bone filling device 160 is introduced through the cannula 153, the smaller diameter portion 162 of the nozzle 163 will desirably extend from the distal tip of the cannula 153 into the targeted bone, while the larger diameter portion 161 (containing the reservoir of bone filling material) desirably remains outside the bone and within the large diameter section of the cannula. Of course, if desired the bone filling device 160 could be used to introduce bone filling material without the use of an associated cannula, as previously described. In addition, if desired, the bone filling device 160 could incorporate an inflatable bladder or o-ring or other sealing mechanism, as previously described, which sealingly engages with the targeted bone to reduce the opportunity for leakage of the filler material.

In an alternative embodiment, the smaller diameter portion 162 is sized such that, when the larger diameter portion 161 abuts the cortical bone of the pedicle, the distal end of the smaller diameter portion extends through the pedicle and emerges into the vertebral body. In this embodiment, the bone filling device could be sized such that, when fully inserted into the cannula, the distal end of the bone filling device would be prevented from contacting and/or breaching the anterior cortical wall of the targeted bone.

A detailed description of similar tools and their associated uses is described in co-pending U.S. patent application Ser. No. 09/695,566, filed Oct. 24, 2000 and entitled “Hand-Held Instruments That Access Interior Body Regions, the disclosure of which is incorporated herein by reference.

IV. A Method of Use

A. Bone Compaction

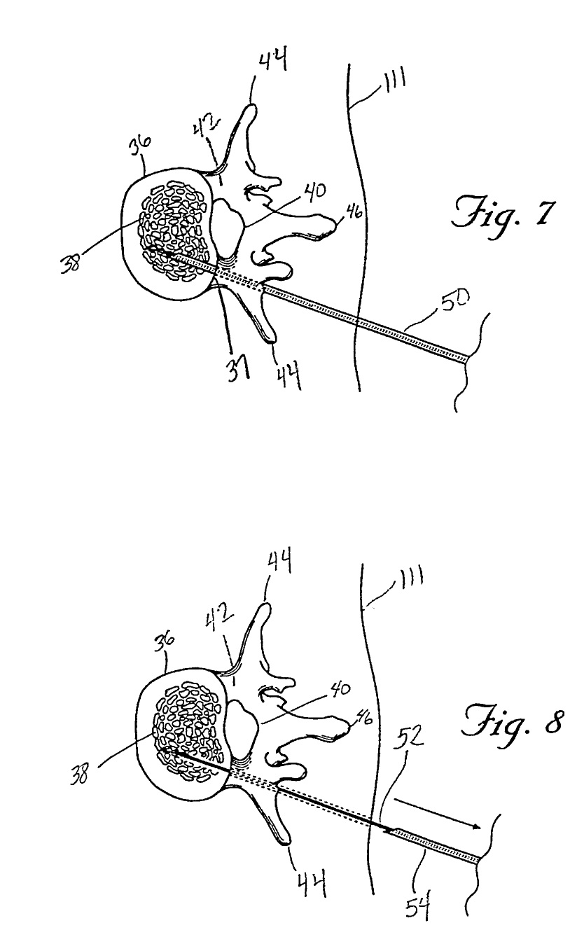

A physician will initially establish an access path through the patient's soft tissue and through the cortical wall 37 of the vertebral body 34. The spinal needle assembly 50 is desirably employed for this purpose (see FIG. 7). In the disclosed embodiment, the physician obtains the spinal needle 54 and inserts the guide wire 52, proximal end 51 first, into the distal end of the spinal needle 54 and passes it through the lumen 56 of the spinal needle 54. The spinal needle assembly 50 is then inserted through soft tissue and through the cortical wall 37 of the vertebral body 34. Alternatively, the physician may first insert a commercially available spinal needle 54 assembly (such as a spinal biopsy needle assembly commercially available from Becton Dickinson & Co.) into the vertebral body 34, remove the stylet from the lumen of the spinal needle, and then insert the guide wire 52 through the lumen 56 of the spinal needle 54 into the vertebral body 34. Of course, in such an arrangement the distal tip of the guide wire would typically be smaller then the interior lumen of the spinal needle.

If desired, the physician may insert the needle directly through the skin, soft tissue and/or bone of the patient, or the physician may create an incision in the skin and/or soft tissue to facilitate insertion of the needle. Once the spinal needle assembly 50 is in a desired position within the vertebral body, the spinal needle 54 is withdrawn (see FIG. 8), leaving the guide wire 52 in place in the cancellous bone 38 of the vertebral body 34. Desirably, the spinal needle 54 will have created a path or opening around the guide wire 52 through the cortical bone 36 and cancellous bone 38 of the vertebral body 34. If desired, a cannulated drill (not shown) may be inserted down the guide wire and into the vertebral body to create and/or increase the size of the opening through the cortical and/or cancellous bone.

After the spinal needle 54 is withdrawn, the bone compaction device 60 is introduced along the guide wire into the vertebral body 34 (see FIG. 9). The central lumen 69 of the bone compaction device 60 desirably passes over the guide wire 52 and into the vertebral body 34. The physician may check the position of the expandable structure 76 by locating radiologically the radio-opaque marker bands 78.

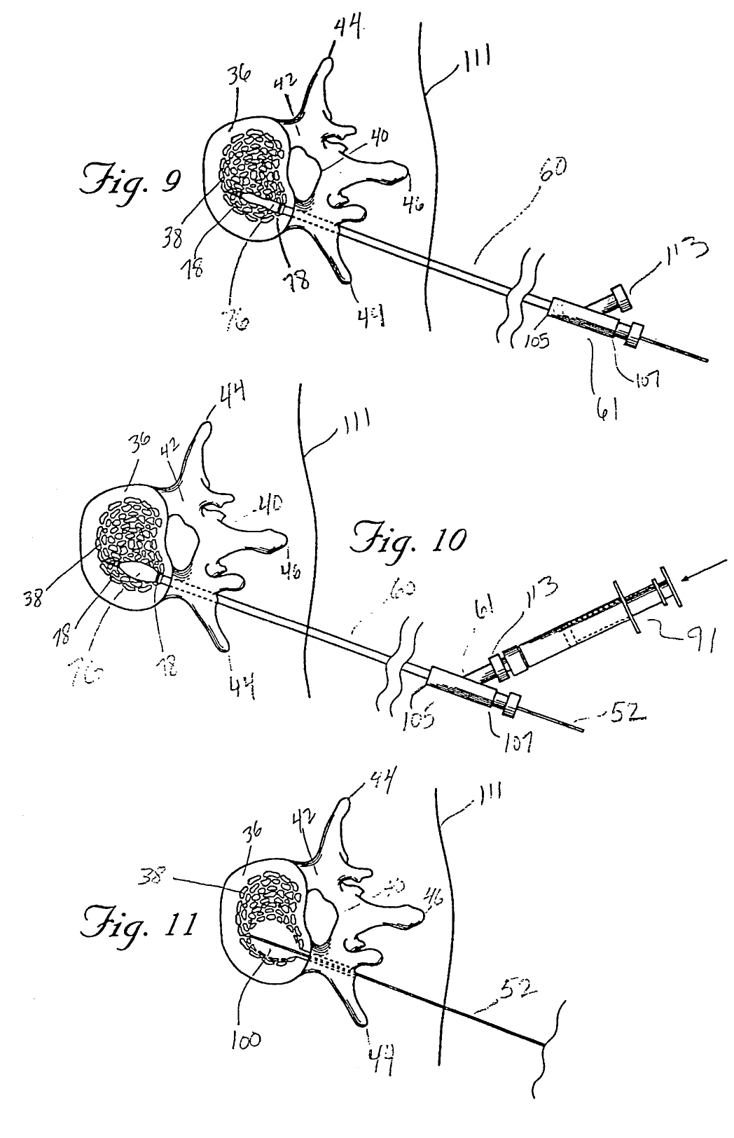

Once it is determined that the expandable structure 76 is properly placed within the vertebral body 34, the physician may introduce expansion fluid 74 into the inflation port 103 of the y-shaped adaptor 61 (see FIG. 10). The expansion fluid 74 passes from the port 103 through the flow passage 68 between the inner catheter tube 66 and the outer catheter tube 64. As the expansion fluid 74 fills the expandable structure 76, it desirably expands the expandable structure 76 (as FIG. 10 shows).

Desirably, the expansion of the expandable structure 76 compresses cancellous bone 38 and/or compacts cortical bone 36, thereby creating an interior cavity 100 (see FIG. 11) within the bone into which a bone filling material 102 may be subsequently introduced. It is desired, and expected, that the expansion results in the lifting or elevating of cortical bone 36 to a more desired position, such as at or near the cortical bone's proper anatomic position. After the interior cavity 100 is created, the physician deflates the expandable structure 76 by using the syringe 91 to draw fluid out of the structure 76. The physician may then withdraw the bone compaction device 60 from the patient.

At this point, it may be desirous to introduce a cannula through the soft tissue to establish an access path to the bone, as previously described. Where a flowable material is injected into the bone, this material has the potential for leaking out of the bone and contaminating surrounding tissue. Because the surgical tools access the vertebral body through an opening formed in the cortical wall, the flowable material may flow towards and through this opening and exit the vertebral body. By positioning a cannula around this opening, the cannula can desirably contain any material which exits the vertebral body through the opening.

As previously described, the cannula 90 may be positioned over the guide wire 52 to access the interior cavity 100 in bone. In one embodiment, the cannula may be imbedded into the opening formed in the bone. Alternatively, the outer body 95 of the cannula 90 may incorporate teeth 92 at its distal end 99, allowing the cannula 90 to be secured to the surface of the cortical bone or at the cortical wall 37, instead of being inserted through the cortical wall 37 and into the vertebral body 34. Desirably, the outer body 95 of the cannula 90 is sized to accept the outer body 85 of a smaller bone filling device 80 for purposes of delivering a bone filling material 102 to the interior cavity 100. After removing the guide wire 52 and the inner body 94 from the proximal end 97 of the outer body 95 of the cannula 90, the bone filling device 80 is inserted through the outer body 95 of the cannula 90 and into the targeted interior cavity 100. The bone filling material 102 is desirably introduced at a relatively low pressure through a syringe 91 coupled to the proximal end of the outer body 85 of the bone filling device 80. For example, injection pressures of less than 1000 psi could be used to introduce the material 102, or more desirably pressures less than 500 psi, or even more desirably pressures less than 360 psi, or even more desirably pressures less than 200 psi, or more desirably pressures less than 50 psi. Most desirably, the pressure of the material exiting the distal end of the bone filling device will approximate ambient atmospheric pressure. Upon completion of filling the interior cavity 100, including the injection of bone filler material from one or more bone filling devices, the final bone filling device 80 is withdrawn through the outer body 95 of the cannula 90, and then the outer body 95 of the cannula 90 is withdrawn, completing the procedure.

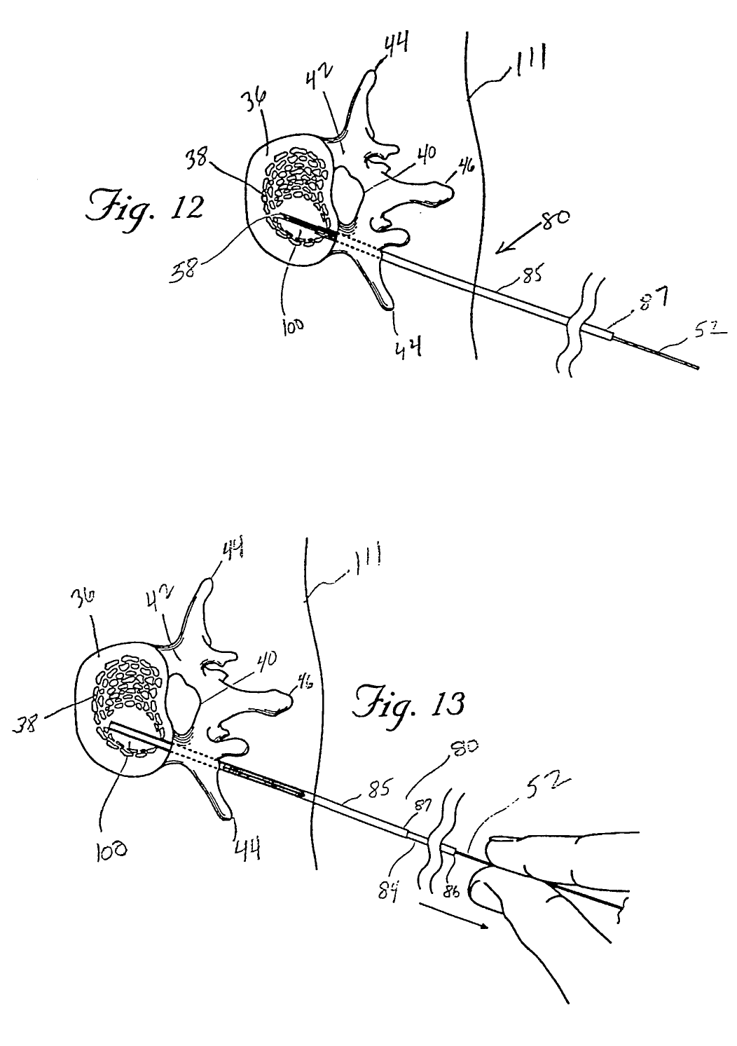

Alternatively, the physician may introduce a bone filling device 80 (see FIG. 12), incorporating an inner body 84 having a distal end 88 and an outer body 85, directly into the interior cavity 100 created in the vertebral body 34 (as FIGS. 6a, 6b, and 6c show). The inner body 84 of the bone filling device 80 is passed over the guide wire 52 and into the vertebral body 34 until the distal surface 53 of the guide wire 52 is reached. Preferably, the distal end 81 of the bone filling device 80 abuts the far side of the interior cavity 100 (as FIG. 12 shows). Desirably, the proximal surface 55 of the distal structure 58 mates with the distal end 88 of the inner body 84. Desirably, the guide wire 52 enables the inner body 84 to center the outer body 85.

The inner body 84 and the guide wire 52 can then be removed from the bone filling device 80 (see FIG. 13). Desirably, the proximal surface 55 of the distal end of the guide wire 52 engages the distal end of the inner body 84, thus aiding in the removal of the inner body 84 from the bone filling device 80.

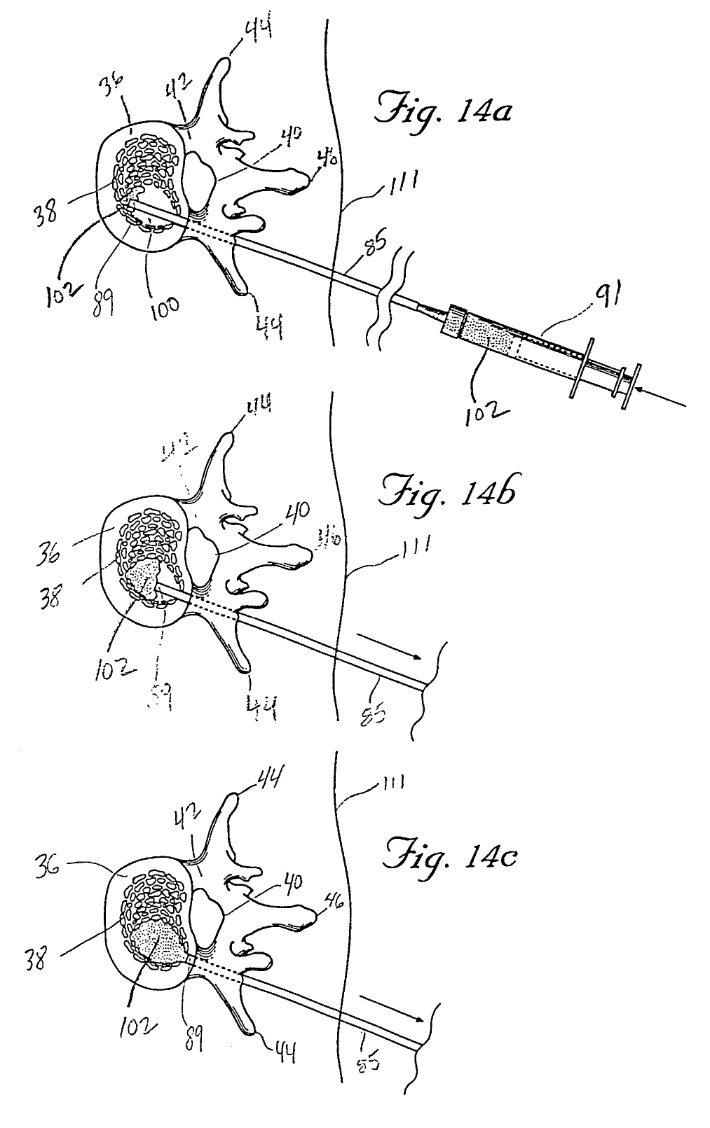

After removing the inner body 84 and the guide wire 52 from the bone filling device 80, the physician may introduce a bone filling material 102 through the bone filling device 80 (see FIG. 14a) and into the interior cavity 100 created in the vertebral body 34. As previously noted, the bone filling material 102 is desirably injected at a relatively low pressure into the targeted vertebral body. As the bone filling material 102 fills the interior cavity 100, the bone filling device 80 can be gradually withdrawn towards the opening in the cortical wall 37 (see FIGS. 14b and 14c). The tamp 106 may be used to urge residual bone filling material 102 into the interior cavity 100. After the interior cavity 100 is substantially filled with bone filling material 102, the bone filling device 80 and tamp 106 are removed.

B. Retrieval of Devices

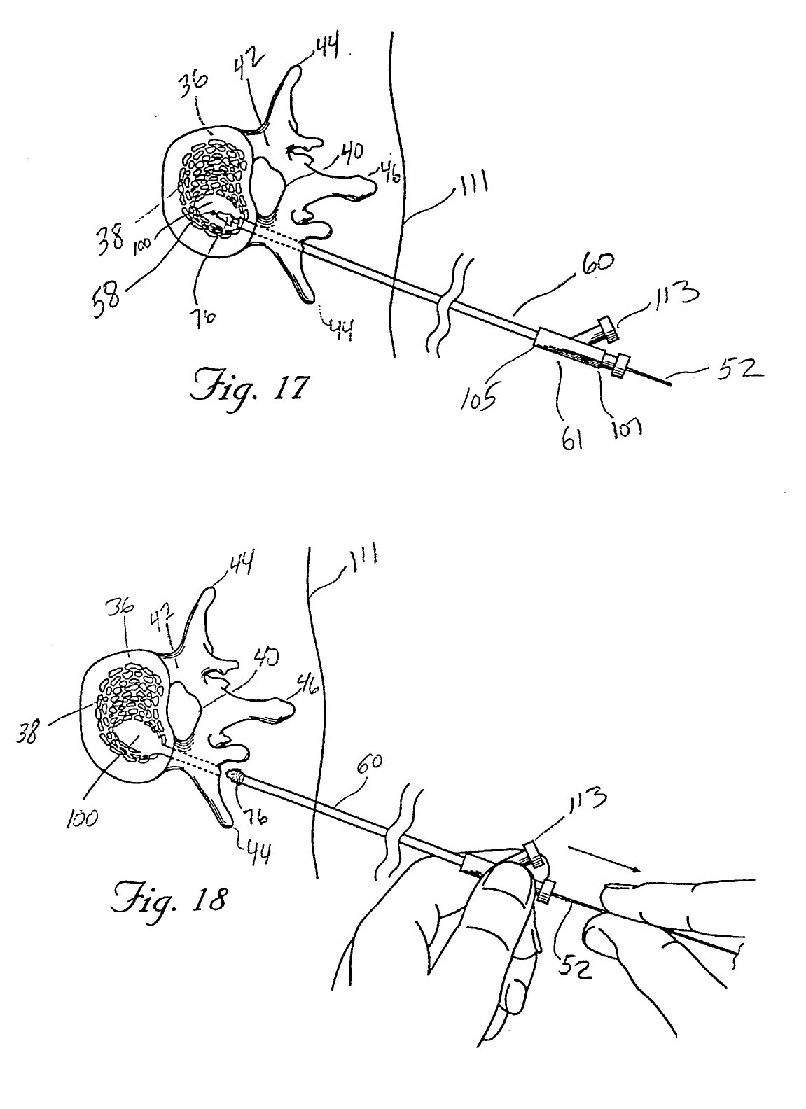

In the event of a failure of the expandable structure 76, such as a tear in the expandable structure 76, the guide wire 52 can also be used to retrieve the expandable structure 76 (see FIGS. 17 and 18). Because the bone compaction device 60 passes along the guide wire 52, and the distal surface 53 of the guide wire 52 is desirably larger than the lumen 68 of the bone compaction device 60, the physician can pull on the guide wire 52 to remove the expandable structure 76. Desirably, in response to a pulling motion, the proximal surface 55 of the distal structure 58 engages or “catches” the expandable structure 76 to aid in its removal.

For example, if the distal portion of the expandable structure 76 separates from the proximal portion of the expandable structure, such as in a complete radial tear, and the inner catheter tube 66 similarly tears, but the expandable structure 76 remains sufficiently intact, the distal structure 58 of the guide wire 52 may be large and/or strong enough to aid in removing the expandable structure 76.

As already described, the distal structure 58 of the guide wire 52 may also be used to remove the inner bodies 84, 94 of the bone filling device 80 and/or the cannula 90. The proximal surface 55 of the distal structure 58 is desirably adapted to mate with the distal end 88 of the inner body 84. The distal end 88 of the inner body 84 is desirably likewise adapted. In response to a pulling motion by the physician, if the proximal surface 55 of the distal structure 58 partly or fully engages the distal 88 end of the inner body 84, the physician may accomplish removal of the inner body 84 from the bone filling device 80. Likewise, the inner body 94 of the cannula 90 may be so removed.

In addition to the specific uses described above, the cavity-forming devices and methods described herein would also be well-suited for use in treating and/or reinforcing weakened, diseased and/or fractured bones in various locations throughout the body. For example, the disclosed devices and methods could be used to deliver reinforcing materials and/or medications, such as cancer drugs, replacement bone cells, collagen, bone matrix, demineralized calcium, and other materials/medications, directly to a fractured, weakened and/or diseased bone, thereby increasing the efficacy of the materials, reinforcing the weakened bone and/or speed healing. Moreover, injection of such materials into one bone within a body could permit the medication/material to migrate and/or be transported to other bones and/or organs in the body, thereby improving the quality of bones and/or other organs not directly injected with the materials and/or medications.

Other embodiments and uses of the invention will be apparent to those skilled in the art from consideration of the specification and practice of the invention disclosed herein. All documents referenced herein are specifically and entirely incorporated by reference. The specification and examples should be considered exemplary only with the true scope and spirit of the invention indicated by the following claims. As will be easily understood by those of ordinary skill in the art, variations and modifications of each of the disclosed embodiments can be easily made within the scope of this invention as defined by the following claims.

We claim:

1. A device for treating bone comprising:
a guide wire including a distal end having an outside diameter;
a guide wire component extending beyond the distal end having an enlarged outside diameter greater than the outside diameter of the distal end;
an expandable structure, the expandable structure incorporating a lumen sized to accommodate the guide wire; and
at least a portion of a diameter of the lumen being smaller than the enlarged outside diameter of the guide wire component.

2. A device as in claim 1, wherein the guide wire component includes a blunt terminal surface.

3. A device as in claim 1, wherein the guide wire component includes first and second differently contoured surfaces.

4. A device as in claim 1, wherein the guide wire component forms part of the distal end of a spinal needle assembly.

5. A device as in claim 1, further comprising:
a substantially rigid spinal needle assemble having a lumen therethrough sized to accommodate the guide wire.

6. A bone access assembly comprising:
a guide wire;
an outer body having an interior lumen;
an inner body sized to occupy the interior lumen, the inner body having an interior passage sized to pass over the guide wire, the inner body having a distal end; and
the guide wire having an enlarged distal end which engages the distal end of the inner body in response to a pulling motion on the guide wire to move the inner body proximally through the outer body for removal from the interior lumen.

7. A bone access assembly as in claim 6, wherein the outer body includes a proximal end, and further including a fitting to couple the proximal end of the outer body to a source of material to convey the material through the interior lumen after removal of the inner body.

8. A bone access assembly as in claim 6, wherein the outer body comprises a cannula to accommodate passage of an instrument after removal of the inner body.

9. A bone access assembly as in claim 6, wherein the guide wire comprises part of a spinal needle assembly.

10. A bone access assembly as in claim 6, further including another instrument having an interior lumen sized to pass over the guide wire.

11. A bone access assembly as in claim 10, wherein the other instrument includes an expandable body to compact cancellous bone.

12. A system for treating bone comprising
a guide wire, and
an expandable body sized to be inserted into bone over the guide wire and undergo expansion in cancellous bone to compact cancellous bone.

13. A system according to claim 12
further including another instrument sized to pass over the guide wire.

14. A system according to claim 13
wherein the other instrument includes a cannula.

15. A system according to claim 13
wherein the other instrument includes a device for injecting material into bone.

16. A method for treating bone comprising the steps of
deploying a guide wire into bone,
deploying an expandable body over the guide wire into bone, and
expanding the expandable body in bone to compact cancellous bone.

17. A method according to claim 16
further including the step of forming a cavity in cancellous bone by expansion of the expandable body.

18. A method of accessing bone comprising the steps of:
deploying a guide wire into bone;
deploying an expandable structure having a lumen extending therethrough over the guide wire and into bone;
expanding the expandable structure within the bone; and
removing the expandable structure from the bone.

19. A method of accessing bone as in claim 18, wherein the expandable structure comprises an expandable balloon.

20. A method of accessing bone comprising the steps of:
deploying a guide wire having an enlarged distal end into bone;
deploying a bone access assembly over the guide wire and into bone, the bone access assembly comprising an outer body having an interior lumen, an inner body sized to occupy the interior lumen, the inner body having an interior passage sized to pass over the guide wire, and the inner body having a distal end; and
removing the inner body proximally from the outer body by pulling on the guide wire to bring the enlarged distal end into engagement with the distal end of the inner body, the interior lumen of the outer body forming a bone access passage.

21. A method of accessing bone as in claim 20 further including the step of:
coupling the bone access passage to a source of material; and
conveying the material through the bone access passage.

22. A method of compacting bone, comprising the steps of:
deploying a guide wire into bone;
passing an instrument over the guide wire, the instrument including an expandable structure;
expanding the expandable structure to compact cancellous bone and create a cavity; and
removing the instrument.

23. A method of compacting bone as in claim 22, further including the steps of:
deploying a cannula over the guide wire; and
pulling on the guide wire to remove the guide wire from the cannula, thereby creating an access passage into the cavity through the cannula.

24. A method of compacting bone as in claim 23, further including the step of conveying a material through the cannula into bone.

25. A method of compacting bone as in claim 23, further including the steps of:
coupling a proximal end of the cannula to a source of material; and
conveying the material through the cannula into bone.
